SEQUENCE LISTING (6 sequences)

SEQ ID NO: 1 - protein, 219 aa, unidentified
EVQLVESGGGLVQPGGSLRLSCAASGYTFTAYGMNWVRQAPGKGLEWMGWINTYTGESTYADSVKGRFTISADTSKNTAYLQMNSLRAEDTAVYYCARFAIKGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 2 - protein, 219 aa, unidentified
DIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGTGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 219 aa, unidentified
EVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 4 - protein, 219 aa, unidentified
DIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 219 aa, unidentified
EVQLQQSGPGLVQPGGSVRISCAASGYTFTAYGMNWVRQAPGKGLEWMGWINTYTGESTYADSFKGRFEFSLDTHNSSAYLQIQSLREEDTAVYYCARFAIQGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 6 - protein, 219 aa, unidentified
DIQMTQSPSSLSASVGDRVTITCKSTKSLLHSNGITYLYWYQQKPGSAPKLLIYQMSHLASGVPSRFSSSGSGTDFTLTISSLQPEDFATYYCAQNLEIPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0339564 A1
(43) Pub. Date: Nov. 6, 2025

(54) EPITHELIAL CELL ADHESION MOLECULE (EPCAM)-FLUORESCENCE IMAGING MOLECULE FOR THE DETECTION OF GASTROINTESTINAL TUMORS AND CANCER POSITIVE LYMPH NODES

(71) Applicant: Academisch Ziekenhuis Leiden h.o.d.n. LUMC, Leiden (NL)

(72) Inventors: Alexander Lucas VAHRMEIJER, Leiden (NL); Cornelis Frederikus Maria SIER, Leiden (NL); Petrus Johannes Karel KUPPEN, Leiden (NL)

(21) Appl. No.: 18/854,229

(22) PCT Filed: Apr. 6, 2023
(86) PCT No.: PCT/NL2023/050181

§ 371 Date: Oct. 4, 2024

Related U.S. Application Data
(60) Provisional application No. 63/328,251, filed on Apr. 6, 2022.

(51) Int. Cl. A61K 49/00; A61B 1/04; A61P 41/00

(52) U.S. Cl. CPC A61K 49/0058; A61B 1/043; A61K 49/0032; A61P 41/00

(57) ABSTRACT

Herein is provided a novel molecule and infusion solutions containing the molecule useful for the detection and imaging of EpCAM positive tumors as well as lymph nodes, such as, but not limited to, lower GI (i.e. colorectal) and upper GI (i.e. gastric and esophageal) cancers. The molecule is comprised of an antibody or antibody fragment capable of specifically targeting EpCAM conjugated to a NIR fluorescent dye. A preferred antibody fragment is VB5-845D and a preferred NIR fluorescent dye is IRDye 800CW. Aspects of the invention provide a method of intraoperative tumor visualization of EpCAM expressing primary tumors and lymph nodes.

### BACKGROUND

Gastrointestinal (GI) cancers, such as colorectal, gastric and esophageal cancer, are amongst the tumors with the highest incidence and mortality rates worldwide (1). Despite improvements in tumor therapies (e.g. neoadjuvant chemoradiotherapy, immunotherapy and targeted therapy), GI cancers are still characterized by high recurrence rates and moderate prognosis, particularly for gastric and esophageal cancer (2). Achieving curation in these patients is primarily dependent on the completeness of tumor resection during surgery. R0 resections (microscopically tumor-negative resection margins) remains the foremost treatment for GI cancers as it improves the local 5-year relapse rates significantly (2, 3). During surgery, surgeons mainly rely on pre-operative imaging modalities to predict the localization of the diagnosed tumor and hence the extension of tissue to be resected. In recent years, neoadjuvant therapy has widely been introduced in the treatment of GI cancers to downstage the tumors before surgery, as it has proven to decrease recurrence rates (4-9). However, neoadjuvant therapy can make accurate margin discrimination challenging, as the visual distinction between vital malignant cells and chemoradiotherapy-induced necrosis or fibrosis can become difficult and hinder accurate tumor localization. Equally, the infiltrative nature of malignant tissue, in, for example, advanced-stage cancers, can make radical resections more challenging (10).

Near-infrared (NIR) fluorescence imaging with tumor-targeted tracers is an evolving technique which offers a strategy to assist surgeons in delineating malignant tissue. Due to its potential of selectively accentuating malignant tissue in real-time, it can be particularly beneficial in patients with advanced-stage cancer or after neoadjuvant therapy for better tissue distinction (11). NIR fluorescence imaging has already been widely investigated and implemented in lymph node mapping, enhancement of vital structures (e.g. biliary tract and ureters) and for intraoperative visualization of different tumor types (12-18). Tumor-targeted fluorescent agents are slowly emerging, where tumor targets such as the carcinoembryonic antigen (CEA) and VEGF (targeted by bevacizumab) have previously been investigated in colorectal cancer imaging (14, 19, 20).

Incomplete tumor removal during oncologic gastrointestinal (GI) resections is associated with higher recurrence and mortality rates. Because neoadjuvant chemo-radiotherapy has become a standard treatment modality for GI cancers, subsequent downstaging makes identification of the primary tumor vs. healthy or scar tissue during surgery even more challenging. Near infrared (NIR) fluorescence imaging can aid surgeons in delineating tumors by selectively enhancing malignant tissue in real-time. The Epithelial Cell Adhesion Molecule (EpCAM) is overexpressed in epithelial cancers, making it a promising target for GI cancer imaging. Thus there remains a need for reliable GI cancer imaging molecule and methods.

### SUMMARY OF THE INVENTION

Herein is provided a novel molecule and infusion solutions containing the molecule useful for the detection and imaging of EpCAM positive tumors as well as lymph nodes, such as, but not limited to, lower GI (i.e. colorectal) and upper GI (i.e. gastric and esophageal) cancers. The molecule is comprised of an antibody or antibody fragment capable of specifically targeting EpCAM conjugated to a NIR fluorescent dye. A preferred antibody fragment is VB5-845D. VB5-845D is an anti-EpCAM antibody sc-Fab fragment for targeting EpCAM-positive tumor cells developed and provided by Sesen Bio (See U.S. Pat. No. 9,822,182, which is herein incorporated by reference). A preferred NIR fluorescent dye is IRDye 800CW, a near infrared fluorescent dye (800 nm) developed by LI-COR Bioscience. The conjugated molecule, consisting of the sc-Fab and IRDye 800CW may be referred to herein as “VB5-845D-800CW.”

Aspects of the invention provide a method of intraoperative tumor visualization of EpCAM expressing primary tumors and lymph nodes, the method comprising intravenous administering to a patient (human or animal) an infusion solution comprising VB5-845D-800CW of the invention prior to surgery at a suitable dose and then allowing sufficient time for the molecule to travel to the tumor. Then the tumor is examined for the presence of the VB5-845D-800CW. In a human patient, preferably the infusion solution is administered at least 3 days prior to surgery. In some embodiments, the infusion solution is administered 5-6 days prior to surgery.

Aspects of the invention provide a method for tumor visualization of EpCAM expressing tumors, the method comprising the topical administration of the molecule according to the present invention or composition according to present invention to a resected tumor.

Another aspect of the invention provides, a method for tumor visualization of EpCAM expressing tumors, the method comprising administering the molecule of the present invention or composition according to the present invention to the gastrointestinal tract.

Another aspect of the invention provides, a method for visualization of EpCAM in circulating cells or fragments in blood, the method comprising the intravenous administration of the molecule of the present invention or composition of the present invention to a patient.

### BRIEF DESCRIPTION OF THE FIGURES

FIG. 1A provides a table showing the subject demographics of the patient study. The patient study included 12 oncologic patients who were administered 6 mg (n=4) or 18 mg (n=8) VB5-845D-800CW at least 3 days prior to surgery (range 3-6 days) to enable fluorescence imaging of colorectal, gastric or esophageal cancer. In the surgical setting, intraoperative and back table fluorescence imaging were performed with the Quest Spectrum Platform. At the pathology department, ex vivo fluorescence imaging was performed on the resected specimen with the Pearl Impulse imaging platform.

FIG. 1B provides data from regarding Mean fluorescence intensity (MFI) shown in the tumors. Subjects were dosed with 6 mg (n=4) or 18 mg (n=8) VB5-845D-800CW at least 3 days prior to surgery (range 3-6 days). A higher dose led to an increase in absolute tumor and normal mucosa MFI. The TBRs were comparable between patients receiving 6 mg or 18 mg VB5-845D-800CW 3-4 days prior to surgery. A longer injection time window of 5-6 days in the 18 mg group resulted in higher TBRs, possibly due to the longer clearance time. The circled subject numbers received VB5-845D-800CW 5-6 days prior to surgery: Subjects 18.2 and 18.7 had a pathologic complete response and an ex vivo TBR of 1.0 (marked grey).

FIG. 1C provides data from regarding ex vivo tumor-to-background ratio (TBR) overview per subject. Subjects were dosed with 6 mg (n=4) or 18 mg (n=8) VB5-845D-800CW at least 3 days prior to surgery (range 3-6 days). A higher dose led to an increase in absolute tumor and normal mucosa MFI. The TBRs were comparable between patients receiving 6 mg or 18 mg VB5-845D-800CW 3-4 days prior to surgery. A longer injection time window of 5-6 days in the 18 mg group resulted in higher TBRs, possibly due to the longer clearance time. The circled subject numbers received VB5-845D-800CW 5-6 days prior to surgery: Subjects 18.2 and 18.7 had a pathologic complete response and an ex vivo TBR of 1.0 (marked grey).

FIG. 2A Intraoperative fluorescence in colorectal cancer during open surgery after administration of 18 mg VB5-845D-800CW 5-6 days prior to surgery.

FIG. 2B Intraoperative fluorescence in gastric cancer during open surgery after administration of 18 mg VB5-845D-800CW 5-6 days prior to surgery.

FIG. 2C Intraoperative fluorescence in esophageal cancer during open surgery after administration of 18 mg VB5-845D-800CW 5-6 days prior to surgery. These images portray that all three cancer types enabled intraoperative tumor fluorescence. The intraoperative tumor-to-background ratio (TBR) in these three tumors were comparable, ranging from 1.8-2.0. Images were obtained with the Quest Spectrum Platform open-field camera.

FIG. 3A provides a graph of the Total immunohistochemistry scores of MOC31 and 323/A3. The TIS was consistently high in the colorectal cancers, where both MOC31 and 323/A3 were strongly expressed. In the upper GI cancers, the TIS varied. No correlation could be made between EpCAM expression and fluorescence, as the mainstream of TBRs were around 2.0, with one outlier of 4.5 (range 1.8-4.5).

FIG. 3B provides accompanying ex vivo fluorescence per subject. The ex vivo fluorescence was measured with the Pearl Impulse imaging platform.

FIG. 4A provides Examples of fluorescence in EpCAM positive and EpCAM negative cancers. FIG. 4A shows an EpCAM positive colorectal tumor (subject 18.6) with vivid fluorescence in the strongly MOC31. The normal mucosa around the tumor shows a much lower EpCAM expression with less intense fluorescence.

FIG. 4B provides Examples of fluorescence in EpCAM positive and EpCAM negative cancers. Figure B shows an EpCAM negative gastric tumor (subject 18.3) with a diffuse mild fluorescent signal.

FIG. 4C provides Examples of fluorescence in EpCAM positive and EpCAM negative cancers. Figure C shows a metastatic lymph node of an EpCAM positive colorectal tumor (subject 18.4) with also a vivid fluorescence pattern and strong EpCAM expression. The tumor area is demarcated by the pathologist with a red line. The fluorescent area on top of the metastasis is necrotic metastatic debris. The fluorescence images were obtained with the Odyssey NIR scanner and the Axio scanner to demonstrate the accuracy of fluorescence on a microscopic level and correlate the localization of fluorescence with the immunohistochemical staining of MOC31 and 323/A3.

FIG. 5A provides a Detailed image of a EpCAM-positive tumor.

FIG. 5B provides a Detailed image of a lymph node. FIG. 5B shows the magnification of the EpCAM positive colorectal tumor (subject 18.6) and metastatic lymph node (subject 18.4) with vivid fluorescence in the strongly MOC31 and 323/A3 expressed tumor. Both specimens show the same distinct pattern of fluorescent epithelium of cylindrical tumor cells, which is reflected by the MOC31 and 323/A3 staining patterns.

FIGS. 6A and 6B provide an electron micrograph displaying extracellular vesicles derived from colon cancer cells, showing a potential target for VB5-845D-800CW. The black and white arrows indicate various sizes corresponding to different types of extracellular vesicles.

FIG. 6C presents a western blot showing the presence of EpCAM and other tumor-associated tumor targets in microvesicles (MV) derived from colon cancer cells.

FIGS. 6D and 6E provide images of tumor cells and microvesicles (TMV) evaluated by fluorescence microscopy. The rationale being that the majority of current liquid biopsy assays evaluating CTC (circulating tumor cells) depend on capturing the neoplastic cells from cancer patients blood via EpCAM targeted antibodies. EpCAM-targeting VB5-845D-800CW, administrated (IV) for the purpose of intraoperative tumor visualization, circulates in the blood for several days, binding to cells located in the tumor, from which some are released in the circulation after invasive growth and extravasation. These cells will be fluorescently stained. Alternatively, VB5-845-800CW will also bind directly to CTCs in the circulation. As a consequence, the isolation of CTC's as a procedure to monitor tumor status before or after operation, is facilitated by the already present fluorescent signal from VB5-845-800CW. Similarly, the recent developments of exploring circulating tumor-cell derived extracellular vesicles, would benefit greatly if the neoplastic vesicles would already be labeled via pre-surgical administration of EpCAM targeting VB5-845.

### DETAILED DESCRIPTION OF THE INVENTION

The Epithelial Cell Adhesion Molecule (EpCAM) is a promising target for fluorescence imaging of multiple tumor types. EpCAM is a transmembrane glycoprotein, involved in homotypic, cell-cell interactions and cell-stroma adhesions, with expression restricted to epithelial cells and differentially expressed between normal tissues and corresponding epithelial malignancies (21-25). Due to its promising multipurpose potential, the inventors developed an EpCAM-specific fluorescent agent, VB5-845D-800CW, to investigate its feasibility in detecting GI tumors during surgery. Immunohistochemistry on human colorectal tissue samples confirmed that EpCAM is a suitable imaging target due to its significantly enhanced expression in tumor tissue in comparison to normal adjacent tissue (26-28). Moreover, the expression of EpCAM does not significantly change after chemoradiotherapy, underscoring its applicability in oncologic patients, as these patients frequently undergo neoadjuvant therapy before surgery (26). Wang et al (29) demonstrated that EpCAM expression in gastric cancer was often correlated to different pathological parameters, such as the Lauren's classification, where the diffuse type often shows higher EpCAM expressions compared to the intestinal type. Based on these preclinical results and existing knowledge on targeting EpCAM, it was hypothesized that the application of NIR imaging with VB5-845D-800CW could aid in real-time detection of GI tumors. Hence, a study was designed for the clinical translation of VB5-845D-800CW, that commenced with a phase I first-in-human study in healthy volunteers to evaluate the safety, tolerability and pharmacokinetics (PK) of VB5-845D-800CW. Subsequently, a pilot study was performed in oncologic patients to assess its feasibility for the fluorescence imaging and detection of lower GI (i.e. colorectal) and upper GI (i.e. gastric and esophageal) cancers.

The present invention provide a novel fluorescent imaging molecule useful for imaging primary and metastatic tumors (with NIR fluorescent imaging) as well as cancer invaded lymph nodes in EpCAM expressing tumors, such as colorectal, gastric and esophageal cancer. With this molecule, intraoperative tumor visualization in the primary tumor as well as affected lymph nodes is possible. Furthermore, because the invented molecule is also able to bind to EpCAM or single cells or cell-derived fragments, the presence of fluorescent signal in blood from infused cancer patients could serve as a diagnostic tool.

Accordingly the invention provides a novel molecule useful for the detection and imaging of EpCAM positive tumors, such as, but not limited to, lower GI (i.e. colorectal) and upper GI (i.e. gastric and esophageal) cancers. The molecule is comprised of an antibody or antibody fragment capable of specifically targeting EpCAM conjugated to a NIR fluorescent dye. A preferred antibody fragment is VB5-845D. VB5-845D is a humanized single chain Fab anti-EpCAM antibody fragment for targeting EpCAM-positive tumor cells, developed and provided by Sesen Bio (See U.S. Pat. No. 9,822,182, which is herein incorporated by reference). VB5-845D is an sc-Fab that binds to epithelial cell adhesion molecule (EpCAM) comprising a heavy chain having the amino acid sequence as shown in SEQ ID NO: 5 and a light chain having the amino acid sequence as shown in SEQ ID NO: 6.

Other antibodies or antibody fragments capable of specifically targeting EpCAM conjugated to a NIR fluorescent dye can be used. For example, the antibody fragment can be aFab, Fab′, F(ab′)₂, scFv, dsFv, ds-scFv, dimers, minibodies, diabodies, bispecific antibody fragments, non-immunoglobulin scaffolds, multimers, and any combination thereof. In a further embodiment, the invention provides a composition comprising the antibody or antibody fragment and NIR fluorescent dye conjugate in a composition. The composition may include the antibody described herein and a pharmaceutically acceptable excipient, carrier, buffer or stabilizer.

The antibody may have a heavy chain with an amino acid sequence selected from SEQ ID NO: 1, SEQ ID NO: 5; and a light chain with an amino acid sequence selected from SEQ ID NO: 2 or SEQ ID NO: 6.

In certain embodiments, the antibody or fragment comprises the Variable heavy chain of SEQ ID NO: 1, SEQ ID NO: 3, or SEQ ID NO: 5. In certain embodiments, the antibody or fragment comprises the Variable light chain of SEQ ID NO: 2, SEQ ID NO: 4, or SEQ ID NO: 6.

In a further embodiment, the antibody or antibody fragment comprises a heavy chain amino acid sequence selected from SEQ ID NO: 1, SEQ ID NO: 5, and a light chain having amino acid sequence selected from SEQ ID NO: 2 and SEQ ID NO: 6.

In some embodiments, a variant antibody or variant antibody fragment may refer to polypeptides or proteins having at least 90%, or 95% sequence identity of the antibody or antibody fragment of the present invention. That is, having at least 90%, or 95% sequence identity to the aforementioned SEQ ID Nos: (such as SEQ ID NO: 1, 2, 3, 4, 5, and 6).

A preferred NIR fluorescent dye is IRDye 800CW, a fluorescent dye developed by LI-COR Bioscience. Conjugated to the antibody fragment the entire molecule may be referred to herein as “VB5-845D-800CW.”

Provided herein is an infusion solution comprising VB5-845D-800CW and 5% glucose for the visualization of metastatic cancer cells in primary tumors and invaded lymph nodes of esophageal, colorectal and gastric cancer cells or any EpCAM expressing tumor such as breast, head and neck, bladder and peritoneal tumors.

Aspects of the invention provide a method of intraoperative tumor visualization of EpCAM expressing primary tumors, the method comprising intravenous administering to a patient (human or animal) an infusion solution comprising VB5-845D-800CW of the invention prior to surgery at a suitable dose and then allowing sufficient time for the molecule to travel to the tumor. Then the tumor is examined for the presence of the VB5-845D-800CW. In a human patient, preferably the infusion solution is administered at least 3 days prior to surgery. In some embodiments, the infusion solution is administered 5-6 days prior to surgery. VB5-845D-800CW binds to EpCAM on the cell surface and then becomes internalized by the cell. The lag time between administering the infusion solution and the surgery can improve the margins and improve the background noise to tumor ratio.

The inventors found that using infusion delivery, they had to wait at least 48 hours to get a good sharp signal in the tumor. Further, the signal lasted about 4-5 days. Because the signal lasted 4-5 days, this suggests that VB5-845D-800CW was internalized and accumulated in tumor cells. Any non-specific binding is unlikely to be internalized, but instead shed. Thus, the surprisingly long half-life for a molecule below the renal threshold for this molecular weight size, and the time required to maximize non-specific shedding relative to specific accumulation in the tumor cells is probably what determines the best TBR (tumor to background ratio) at 4-5 days. Imaging agents typically rely only on specific binding to tumor cells and fast clearance from circulation. So overall, it was not obvious that the VB5-845D-800CW imaging agent would bind and internalize leading to the accumulation of the 800CW dye in tumor cells overtime.

Aspects of the invention also provide a method for intraoperative visualization of lymph nodes that have been invaded by EpCAM expressing cancers, the method comprising intravenous administering to a patient an infusion solution comprising VB5-845D-800CW of the invention of the invention prior to surgery at a suitable dose and then allowing sufficient time for the molecule to travel to affected lymph nodes. Then the physician examines one or more of the lymph node sections for the presence of the VB5-845D-800CW molecule. In some embodiments, the solution is administered at least 3 days prior to surgery and in some embodiment the solution is administered 5-6 days prior to surgery. The lag time between administering the solution and the surgery can improve the margins and improve the background noise to tumor ratio. Using solutions and method of the invention, microscopic evaluation of metastatic lymph nodes showed recognizable fluorescence patterns of tumor areas as small as 100 μm with EpCAM expression. The method may further comprising after removing a cancerous lymph node, removing and examining a neighboring lymph node and repeating until the neighboring lymph node examined does not contain any detectable cancer cells.

Using the VB5-845D-800CW molecule, the inventors were able to detect metastatic tumor cells in lymph nodes and this detection was more sensitive than other detection mechanisms. They studied lymph nodes that had been determined to be tumor positive. These lymph nodes were microscopically sectioned. Since metastasis in lymph nodes show a diffuse pattern, it is often the case that certain sections of the lymph node will not contain any cancer cells even though cancer cells are present in other sections of the lymph nodes. This could lead to issues of mistakenly identifying cancerous lymph nodes as noncancerous. Using the VB5-845D-800CW molecule, cancer cells were detectable in lymph node sections that had previously been indicated as not having cancer cells (even though other sections did contain cancer cells). Thus, it may be possible that using the VB5-845D-800CW molecule, a cancerous lymph node can be detected that would have been missed using prior methods of detection.

Preferably in the methods above, the interoperative surgery is open oncologic resections because during minimal invasive surgery, due to a lack of sensitivity of the current NIR minimal invasive camera systems. However, as NIR camera and lighting systems improve, the methods could be used in endoscopic procedures.

One skilled in the art would determine the optimum dosing of the molecule of the invention in the infusion solution. Generally, the infusion solution is administered at a dose of 6 mg to 18 mg in a human adult patient.

Aspects of the invention also provide a method of removing esophageal, colorectal and gastric cancerous tissues (or any EpCAM expressing tumor such as breast, head and neck, bladder and peritoneal tumors), the method comprising administering an infusion solution of the invention to a patient, waiting for a time period to allow the solution to travel and migrate into the tumor and/or lymph nodes, and then visualizing the cancerous tissue for the presence of the VB5-845D-800CW molecule in the tissues with fluorescence during open surgery and then removing the visualized tumor tissue. The time period between administration of the solution and the surgery is to allow the fluorescent molecule of the invention to travel to the tumor and/or lymph nodes. The time can depend on various factors such as the size of the tumor, the size of the patient, etc. In humans, preferably the time is about 72 hours to 124 hours. In another aspect the invention provides a method of tumor resection by administering the molecule of the present invention to a subject undergoing surgery, obtaining an in situ image of the tissue where the image allows for the detection of a diseased cell, if present in the tissue, removing the diseased cell detected and repeating the imaging and removing step until no diseased cell is detected in the tissue. The diseased cell may be a tumor cell.

The invention also provides a method of determining whether a tumor in a patient expresses EpCAM, the method comprising administering to the patient the infusion solution, waiting for a time period and then measuring fluorescence from the VB5-845D-800CW molecule during open and/or endoscopic surgery to determine if the tumor expresses EpCAM. This method can also be used to determine whether a lymph node has been invaded by an EpCAM expressing cancer.

Also provides is a method of determining whether rectal and esophageal cancer patients after neoadjuvant therapy require surgery, the method comprising administering to the patient the infusion solution, waiting for a time period and then performing endoscopic surveillance (detecting fluorescence from the VB5-845D-800CW molecule) to identify whether there any tumor cells present, and if present determining that surgery is necessary to remove the cancerous tissue.

In addition, the invention provides a method of guiding drug delivery where proliferative areas of a tumor can be delineated and be treated. A patient would receive the infusion solution and the tumor would be located through the NIR fluorescence of the VB5-845D-800CW molecule and then a physician could direct or guide a therapeutic drug to the site of the tumor (either a physical guidance, such as direct injection of the therapeutic into the sight where the fluorescence from the molecule shows up, or a guidance system such as using a therapeutic that only recognizes EpCAM).

The invention provides a method for tumor visualization of EpCAM expressing tumors, the method comprising the topical administration of the molecule according to the present invention or composition according to present invention to a resected tumor. The tumor may subsequently examined for the presences of EpCAM. “Tumor” refers to new tissue containing cancer cells. Tumors can be solid or non-solid tissue, such as those present in the circulatory system, such as leukemia. In a typical solid tumor cancer resection, the surgeon removes the bulk of the tumor.

The molecule or composition may be applied ex vivo by smearing or spraying. The molecule or composition can be spread or smeared across the resected tumor. Ideally the smear is uniform in thickness and length. The term smear as used herein refers to a spread of the molecule over a surface. The term spray as used herein refers to applying the molecule in the form of tiny drops.

The invention provides a method for tumor visualization of EpCAM expressing tumors, the method comprising administering the molecule according to the present invention or composition according to the present invention to the gastrointestinal tract. The tumor may be subsequently examined for the presences of EpCAM. The molecule or composition may be administered by endoscopy. Endoscopy provides a means for physicians to perform medical procedures while observing the patient's internal structure while minimizing patient trauma. Numerous endoscopes have been developed and classified according to specific applications (e.g., cystoscopy, colonoscopy, laparoscopy, upper gastrointestinal endoscopy and other examinations). The endoscope can be inserted into the natural opening of the body, or the endoscope can be inserted through a notch in the skin.

An endoscope is usually a rigid or flexible tubular elongated shaft having a video camera or fiber optic lens assembly at its distal end. The shaft is connected to a handle, which may also include a direct eyepiece. Usually, it can also be confirmed by an external screen. Various treatment tools can be inserted through the working channel of the endoscope to perform various surgical procedures. Endoscopes currently in use (colonoscopes, etc.) typically have anterior cameras for observing organs (colon, etc.), lighting, and fluid injectors for cleaning camera lenses and occasional lighting. It also includes a working channel for inserting a treatment tool (eg, for removing polyps found in the colon). Often, the endoscope is also inserted into a body cavity such as the colon and has a fluid injector (“spout”) to clean the body cavity. A commonly used illumination is an optical fiber that transmits light generated at a distance to the tip of the endoscope. It is also known to use light emitting diodes (LEDs) as lighting.

The invention provides a method for tumor visualization of EpCAM in circulating cells or cell fragments in blood, the method comprising the intravenous administration of the molecule according to the present invention or composition according to the present invention to a patient. The blood may be subsequently examined for the presences of EpCAM via the fluorescent signal derived from the molecule of invention. The circulating cells or fragments maybe an exosome or other extracellular vesicle. Extracellular vesicles are shed from their parent cell and consist of cytosol material wrapped by cell membrane. Extracellular vesicles are produced by cancer cells, mesenchymal cells, thrombocytes, immune cells, platelets, and endothelial cells, and their membranes are believed to contain a similar protein repertoire as the cells from which they arrive. Hence, they may be used as liquid biopsy for diagnosis of the presence of cancer tissue, or the prognosis of the patient, provided that the proper tumor marker is chosen for detection to distinguish them from vesicles from other, non-malignant cells. Most applications, exploring the diagnostic capacity of extracellular vesicles, use the malignancy associated EpCAM protein to isolate and/or recognize tumor derived vesicles, often via an EpCAM binding antibody. Since the invention is administrated into patients and consists of a traceable EpCAM binding antibody fragment, tumor cells and cell fragments derived from the blood these patients may be used to monitor the presence of the tumor in a relatively simple assay. Blood components may be whole blood, red blood cells, hemoglobin, platelets, plasma, or white blood cells.

The invention provides the molecule of the present invention for use in a diagnostic method practiced on the human or animal body. The molecule maybe administered intravenously, topically or by endoscopy.

The invention provides the molecule of the present invention for use in detecting EpCAM expressing primary tumors and/or lymph nodes. The tumor maybe a gastro intestinal tumor. The molecule maybe administered by endoscopy.

The invention provides the molecule of the present invention for use in detecting cancer. The cancer maybe lower gastro intestinal and/or upper gastro intestinal cancer. The gastro intestinal cancer maybe colorectal, gastric or esophageal cancer. The cancer maybe breast, head and neck, bladder or peritoneal tumors. The molecule maybe applied topically on either a resected tumor or a resected tumor bed.

The invention provides the molecule of the present invention for use in detecting EpCAM expressing circulating cells or fragments in blood. The circulating cells or fragments maybe an exosome and/or extracellular vesicle.

### EXAMPLES

### Example 1: Methods

### GMP Production VB5-845D-800CW

VB5-845D is an anti-EpCAM antibody Fab fragment for targeting EpCAM-positive tumor cells developed and provided by Sesen Bio. IRDye 800CW, a fluorescent dye developed by LI-COR Biosciences was used for the NIR fluorescence imaging and delivers enhanced sensitivity due to low background auto-fluorescence in the NIR region. Conjugation of the dye to the anti-EpCAM Fab fragment VB5-845D generated the novel fluorescent imaging agent, designated as VB5-845D-800CW. The EpCAM Fab was conjugated to DOTA and IRDye 800CW using N-Hydroxysuccinimide (HND) ester chemistry against primary amines following manufacturer protocol (Thermo Scientific, USA). The conjugation was performed with a 4-fold molar excess of the NIR dye or DOTA and incubated and mixed for four hours at room temperature after which the non-conjugated dye was removed using a Zeba Spin Desalting column (Pierce Biotechnology, Rockford USA). Manufacture, purification, formulation and quality control were performed at the department of Clinical Pharmacy & Toxicology of Leiden University Medical Centre (LUMC). The drug product consists of VB5-845D-800CW in phosphate buffered saline pH 7.4 solution in glass injection vials and is a solution with a clear blueish-green color. The final product (VB5-845D-800CW) produced for the clinical trial batch was formulated at a concentration of 2 mg/ml, each vial containing 5 ml of the drug product solution (thus 10 mg/vial).

### Healthy Volunteer Study

The phase I first-in-human study was performed at the Centre for Human Drug Research (CHDR) and consisted of 16 healthy volunteers. The primary objective was to assess the safety, tolerability and PK (in serum, urine and skin/mucosa) of VB5-845D-800CW. The design was a randomized, placebo-controlled, double-blinded study where subjects were randomized to receive a single intravenous (IV) dose of VB5-845D-800CW or placebo. Three consecutive dosing cohorts were applied, where the following doses were administered respectively: 1.5 mg (cohort 1), 6 mg (cohort 2) and 18 mg (cohort 3). Subjects were assigned to the cohorts based on the order of enrolment; six subjects in cohort 1, five subjects in cohort 2 and five subjects in cohort 3. In the first cohort, subjects were randomized 4:2 for VB5-845D-800CW and placebo, where a sentinel approach was used in the first two subjects. In cohort 2 and cohort 3, the subjects were randomized 4:1 for VB5-845D-800CW and placebo. The investigator, staff and subjects were blinded with respect to the treatment until the end of the study. Placebo consisted of 5% glucose and was formulated and packaged identically to VB5-845D-800CW. VB5-845D-800CW and placebo were administered via an IV infusion drip over 30 minutes. Subjects were admitted for 24 hours, where routine laboratory tests, electrocardiograms (ECG), vital signs and adverse events (AE) were recorded for safety assessments. At fixed time points following IV administration, blood and urine samples were collected and fluorescence measurements were done for PK analysis. Blood samples were collected predose and immediately after administration start at 2, 6, 15, 30 and 45 minutes as well as 1, 2, 4, 8, 12 and 24 hours, 2 days, 4 days and 10 days. Timed urine specimens were taken from the following time windows postdose in all three cohorts: 0-2 hours, 2-4 hours, 4-8 hours, 8-12 hours and 12-24 hours. Fluorescence in skin (dorsal side of the foot) and mucosa (inner part of the bottom lip) were measured using the Quest Spectrum Platform (Quest Medical Imaging, Middenmeer, the Netherlands). Blood and fluorescence intensity were measured up to two days in cohort 1, four days in cohort 2 and ten days in cohort 3. Additional measurements were added to the later cohorts since a relatively long half-life was perceived in the preliminary data after each respective cohort. Skin and mucosa fluorescence were measured by calculating the signal-to-baseline ratio (SBR) in each image using ImageJ 1.51j8 (National Institute of Health, MD, USA). The pre-dose image was used as the baseline measurement. A region of interest (ROI) was drawn around fluorescence and divided by the baseline signal of the ROI to create an SBR. After completion of the healthy volunteer study, the obtained results were used to determine the dose range and injection time window for performing intraoperative imaging in the subsequent feasibility study in patients with GI cancer.

### Patient Study

The study in patients was an open-label exploratory study performed at the Leiden University Medical Centre (LUMC) and included 12 oncologic patients diagnosed with lower GI or upper GI cancer. The primary objective was to evaluate the feasibility and efficacy of tumor detection using VB5-845D-800CW. Two dose levels comprising of 6 mg and 18 mg were evaluated. VB5-845D-800CW was administered via an infusion drip over 30 minutes and the patients then admitted for up to six hours for observation and safety measurements (i.e., AE recordings, ECG and vital signs). All patients were administered VB5-845D-800CW at least three days prior to the scheduled surgery (range 3-6 days). Injection time windows (time interval between administration and surgery) were divided into two timing schemes; 3-4 days prior to surgery and 5-6 days prior to surgery. After IV administration, blood samples were obtained for supplementary PK analysis. Assignment to dosage group was based on the order of enrolment. At least three patients had to be included in the 6 mg dose group before escalation to 18 mg could occur. Intraoperative imaging during the surgical procedures was performed with the Quest Spectrum Platform and Olympus NIR system (CLV-S200-IR; the Netherlands). The Quest Spectrum Platform is a dedicated NIR camera system for open and minimal invasive (laparoscopic) surgery. The Olympus NIR system only allows fluorescence imaging in a minimal invasive setting. Fluorescence imaging was performed during initial inspection of the abdomen, after dissection (before tumor resection), and after tumor resection. Tumor fluorescence was determined by calculating the tumor-to-background ratio (TBR) using ImageJ 1.51j8. A ROI was drawn around tumor fluorescence and one around the surrounding background area (30). The fluorescence quotient of the tumor and background signal constituted the intraoperative TBR. After surgical resection, back table imaging of the specimen was performed with the Quest Spectrum Platform, using the open-field camera mount. This was done to compare the intraoperative abdominal tumor fluorescence intensity with the back-table fluorescence outside the patient. Afterwards, the tissue specimen was analyzed by the pathologist as described below.

### Pharmacokinetics (PK) and Statistical Analysis

The healthy volunteer study was designed using a commonly accepted number of subjects per group. For the patient study, the sample size was not based on statistical power considerations due to the exploratory nature of the study. VB5-845D-800CW concentrations in blood and urine were estimated from calibration curves in human whole blood and phosphate-buffered saline (PBS, pH 7.4) using the Pearl Impulse imaging platform (LI-COR Biosciences, NE, USA). PK results were analyzed with software dedicated for PK analysis (R 2.12.0 for Windows) using non-compartmental methods. Data are summarized in graphs and bar charts generated by GraphPad Prism (version 8.0).

### Pathology and Ex Vivo Tumor Imaging

Resected tumor specimens and lymph nodes were examined and revised by a GI pathologist. Before the specimen was processed for histopathological analysis with standard haematoxylin-eosin (H&E) staining of tissue sections, the bread-loafed tumor specimen (gross macroscopy) was imaged with the Pearl Impulse imaging platform. For each patient, the mean fluorescence intensity (MFI) of the primary tumor and normal tissue were measured with the integrated instrument software ImageStudio Ver 5.2 (LI-COR Biosciences). The following formula was then used to calculate the ex vivo TBR=MFI tumor/MFI normal tissue.

### Immunohistochemistry

Formalin-fixed paraffin embedded (FFPE) tumor, adjacent normal tissues and harvested lymph nodes were sectioned at 4 μm thickness for additional immunohistochemistry staining with the mouse monoclonal antibodies MOC31 (Acris Antibodies) and 323/A3 (Pathology, LUMC) to assess the presence of EpCAM. For each patient, two representative tumor tissue sections and two normal tissue sections were analyzed for EpCAM expression. First, the FFPE sections were deparaffinized with xylene and rehydrated in ethanol solutions (100%, 70% and 50%). Endogenous peroxidase activity was blocked with 0.3% hydrogen in demineralized water for 20 minutes. An antigen retrieval step utilizing heat induction (95° C.) using PT Link (pH Low (citrate buffer pH 6.0), DAKO) was performed for MOC31 antibody. For 323/A3, slides were placed in a water bath (37° C.) and incubated with 0.1% trypsin for 30 minutes. The sections were incubated overnight with primary antibody (diluted in 1% BSA/PBS) at room temperature. After three washing steps with PBS, incubation with HRP-labelled secondary mouse antibody (DAKO) was performed for 30 minutes. A positive internal control was present, and the negative control was incubated with 1% BSA/PBS. The binding of the antibody was visualized using substrate DAB (3,3-diaminobenzidine) (DAKO). Sections were counterstained with haematoxylin for 15 seconds, dehydrated in stove (37° C.) for 30 minutes and mounted with pertex (Histolab).

Evaluation of immunohistochemistry staining was performed independently by two observer pairs. All tissue sections were scored for MOC31 and 323/A3 staining. The total immunostaining score (TIS) was calculated for each respective antibody by multiplying the percentage score (PS) and intensity score (IS) found. The PS represented the percentage of positively stained cells and ranged between 0 and 4 (0: none/1: <10%/2: 10-50%/3: 51-80%/4: >80%). The IS represented the intensity of the stained cells and ranged between 0 and 3 (0: no staining/1: mild/2: moderate/3: intense). The calculated TIS was then defined as negative (0-1), mild (2-3), moderate (4-8) or strong (9-12). Additionally, the tissue sections were scanned with the Odyssey NIR scanner (LI-COR Biosciences) and the high-resolution ZEISS Axio Scan.Z1 NIR-scanner to compare microscopic fluorescence and morphological fluorescence patterns of primary tumors with the metastatic lymph nodes.

### Ethics Committee

The studies were conducted in accordance with the principles of the Helsinki Declaration of 1975 (as amended in Tokyo, Venice, Hong Kong, Somerset West, Edinburgh, Washington, and Seoul), and the laws and regulations of the Netherlands. The study was approved by a certified medical ethics review board (Stichting BEBO, Assen, the Netherlands). All subjects provided written informed consent prior to the start of any study-related procedures. The study is registered in the European Clinical Trials Database under number 2018-002859-15, as well as in the Netherlands Trial Register under ID NTR7570.

### Example 2: Results

A total of 28 subjects (16 healthy volunteers and 12 patients) were enrolled. The healthy volunteers consisted of four males and 12 females, with a median age of 22 years (range 19-55 years). The patient study consisted of 12 oncologic patients (eight males and four females) with a median age of 62 years (range 46-74 years) diagnosed with either lower GI (n=6) or upper GI (n=6) cancer, scheduled for an open (n=5) or minimal invasive laparoscopic (n=7) oncologic resection. Table 1 displays the subject demographics for the patient study, including the details of diagnosis, neoadjuvant treatment, surgical procedures and tumor histology.

### Safety and Tolerability

There were no serious adverse events (SAE) recorded. Adverse events (AE) reported during the study were either mild or moderate, none required interruption of the study and all resolved without sequalae. Within the healthy volunteer study, three subjects reported a headache after VB5-845D-800CW administration; two were assessed as possibly related to VB5-845D-800CW and one as unrelated (placebo). In the patient study, seven patients experienced a total of 16 AEs. Only one of these AEs (warm feeling) was assessed as possibly related to VB5-845D-800CW as it was noted shortly after start of administration and disappeared at the end of infusion. The remaining 15 AEs (e.g. procedural nausea, vomiting, abdominal pain, fever, urinary tract infection, hypotension, dyspnea) were all judged as unrelated to VB5-845D-800CW as they occurred after surgery and were associated with the post-operative course of the undertaken procedure. Furthermore, no trends or changes of clinical importance were observed in the vital signs, clinical laboratory tests or ECGs after dosing in all subjects. Overall, all the administered doses were well tolerated. Importantly, doses up to 18 mg did not elicit any acute toxicity, nor any immune-related reactions.

### Pharmacokinetics

A non-compartmental analysis (NCA) was performed on the PK data. Since the doses 6 mg and 18 mg were administered to patients, these NCA results are described. For both these doses, the maximum blood concentrations were achieved immediately at the end of infusion. In the 6 mg dose, the concentration declined with a mean apparent half-life of 46.8 h, had a mean clearance rate of 0.06 L/h and a mean distribution volume of 3.7 L. In the 18 mg dose, the concentration declined with a mean apparent half-life of 73 h, had a mean clearance rate of 0.06 L/h, and a mean distribution volume of 5.9 L. The PK profiles in blood were similar in both the healthy volunteers and patients. In the healthy volunteers, an average cumulative urine excretion (expressed as the percentage of the injected dose) of 63% was observed 24 h post dose in the 6 mg cohort, while this was 48% in the 18 mg cohort. The fluorescence in skin and mucosa in the healthy volunteers showed a SBR increase as the dose increased. In mucosa the highest SBR was measured approximately 4 hours post-dose with an average SBR of 1.4 (1.5 mg), 10.9 (6.0 mg) and 25.5 (18 mg). In skin the highest SBR was measured approximately 24 hours post-dose, with an average SBR of 1.4 (1.5 mg), 5.0 (6 mg), and 16.2 (18 mg). In mucosa, a higher SBR was perceived when compared to the skin. In the 18 mg cohort, fluorescence was measured up to 10 days after IV administration, where fluorescence was fading back to baseline.

### Tumor Detection

Of the 12 surgical procedures, five patients underwent an open resection, that included gastric resections (n=3), a trans-hiatal esophageal resection (n=1) and a sigmoid resection (n=1). In four of these open resections, tumor fluorescence was visible in the surgical setting (intraoperatively and on the back table) with the open system of Quest Spectrum Platform. There was no fluorescence visible in the surrounding organs, such as the liver and intestines. The one patient without tumor fluorescence (intraoperatively and on the back table) had a pathologic complete response (pCR) after neoadjuvant therapy, confirmed by histopathology.

Seven patients underwent a minimal invasive resection, which included laparoscopic low anterior resections (n=2), laparoscopic hemicolectomies (n=2), laparoscopic transthoracic oesophageal resections (n=2) and a laparoscopic sigmoid resection (n=1). In all laparoscopic procedures (with both the Quest Spectrum Platform and Olympus NIR system), no intraoperative tumor fluorescence was visible. However, tumor fluorescence was apparent on the back table with noticeable tumor demarcation in six of these seven patients. The one patient without back table fluorescence had a PCR after neoadjuvant therapy, which corresponded to histopathology.

### Tumor Fluorescence

The first four patients received 6 mg VB5-845D-800CW. Since no fluorescence was visible during minimal invasive procedures in the 6 mg dose, the following patients received 18 mg to obtain a fluorescent signal in the minimal invasive setting. However, a higher dose had no effect on the intraoperative fluorescence. In vivo fluorescence was only visible during open procedures due to differences in sensitivity between the open and minimal invasive imaging system.

The available intraoperative TBRs (measured in the open procedures) were similar (for both dose levels, up to 6 days in the high-dose cohort. In the 6 mg dose, 1/1 (100%) demonstrated an intraoperative TBR measurement of 1.8 for an upper GI tumor. In the 18 mg dose, intraoperative TBRs were measured in four patients. Two patients with upper GI cancers showed an intraoperative TBR of 2.0 and third patient with a lower GI cancer showed an intraoperative TBR of 1.8. One intraoperative TBR was 1.1 measured in the case with a PCR.

The back table TBRs were also comparable in both dose levels. In the 6 mg dose, the average back table TBR for the lower GI and upper GI cancers were 1.6 (SD±0.21) and 1.8 (SD±0.07), respectively. In the 18 mg dose, the average back table TBR for the lower GI and upper GI cancers were 1.7 (SD±0.31) and 1.9 (SD±0.00), respectively. Increasing the dose of VB5-845D-800CW did not lead to significantly higher intraoperative or back table TBRs (p=0.53).

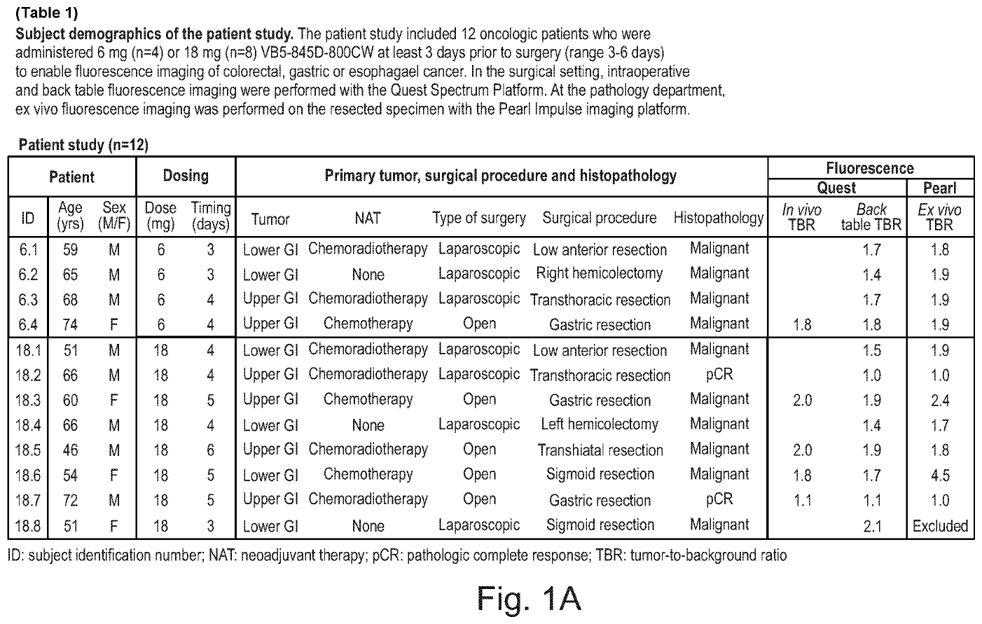

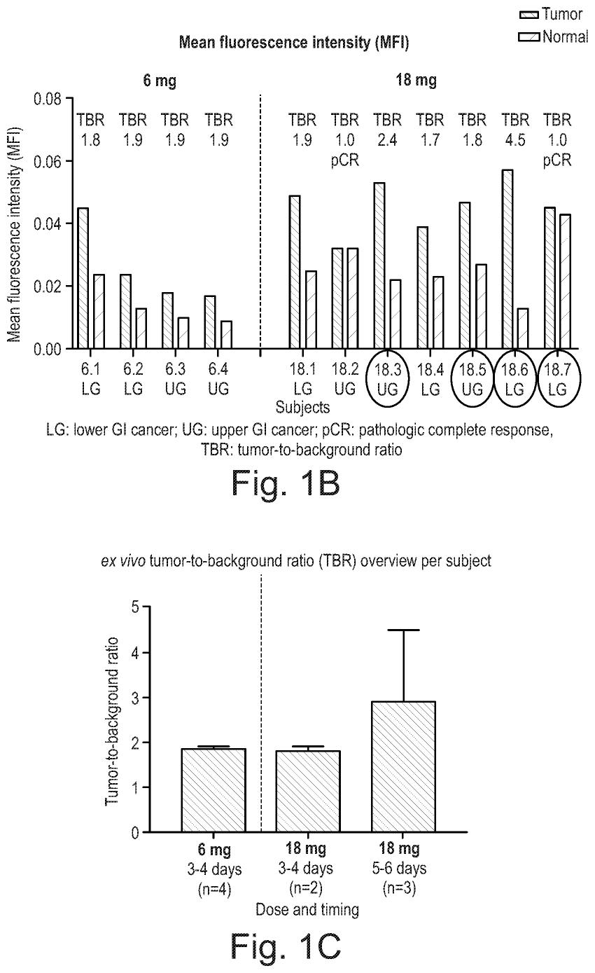

The MFI, measured in tumor and normal mucosa during the ex vivo analysis with the Pearl Impulse imaging platform, showed a difference between the two dose levels. As the dose increased, the MFI in tumor and normal mucosa increased (FIG. 1A). In the 6 mg dose, all patients were dosed 3-4 days prior to surgery (n=4). In the 18 mg dose, the patients were dosed either 3-4 days (n=4) or 5-6 days (n=4) prior to surgery. One patient, dosed with 18 mg, 3 days prior to surgery, was missing ex vivo results and was excluded in the ex vivo analysis. It was apparent that a higher dose led to an increase in tumor and mucosa MFI. Yet, the timing of administration appeared to have an influence on the TBR. An injection time window of 3-4 days prior to surgery showed no difference in ex vivo TBR between the two dose levels (1.9 in 6 mg vs. 1.8 in 18 mg). However, when a longer injection time window of 5-6 days was implemented in the higher dose, the mean TBR increased to 2.9 (SD±1.4) (FIG. 1A) suggesting that time rather than dose, for the doses tested, was the critical determinant for visualizing fluorescence.

### Pathology and Immunohistochemistry

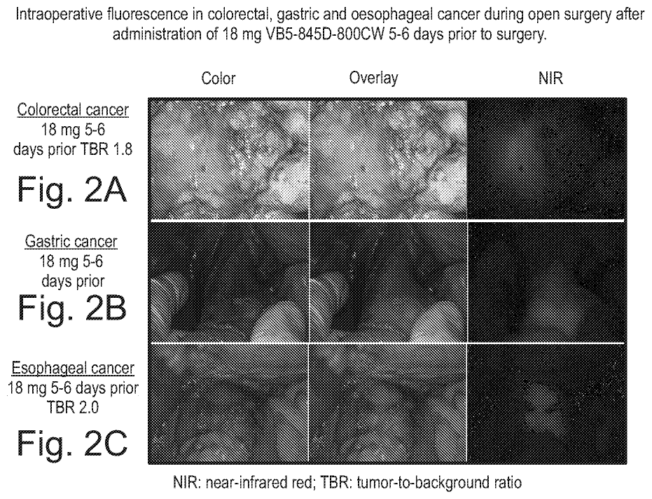

All colorectal, gastric and esophageal cancer tissues with vital malignant cells, displayed fluorescence with VB5-845D-800CW (FIGS. 2A, 2B and 2C). Despite the high dose of 18 mg, the two PCR cases without malignant cells, as confirmed by histology, did not show fluorescence resulting in ex vivo TBRs of 1.0.

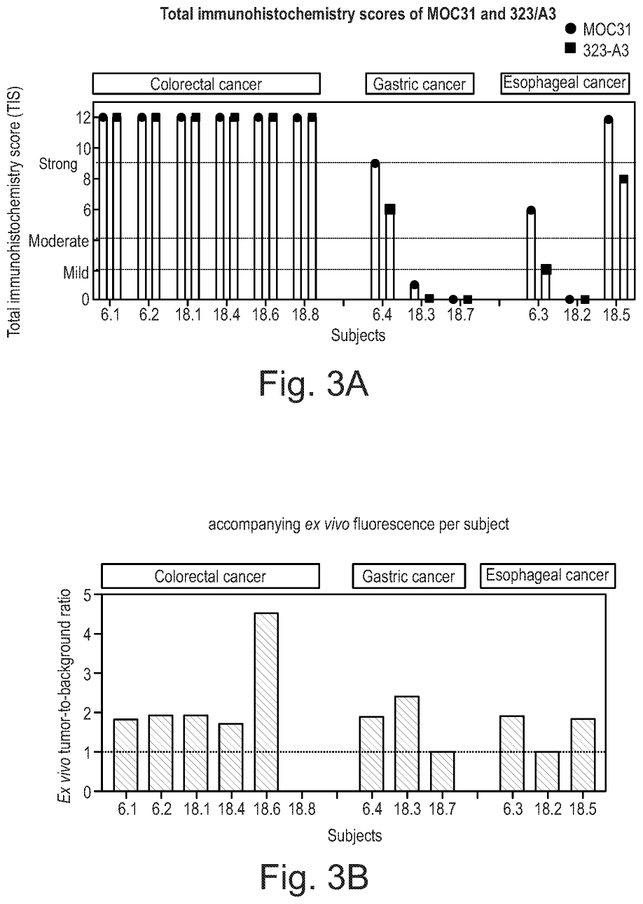

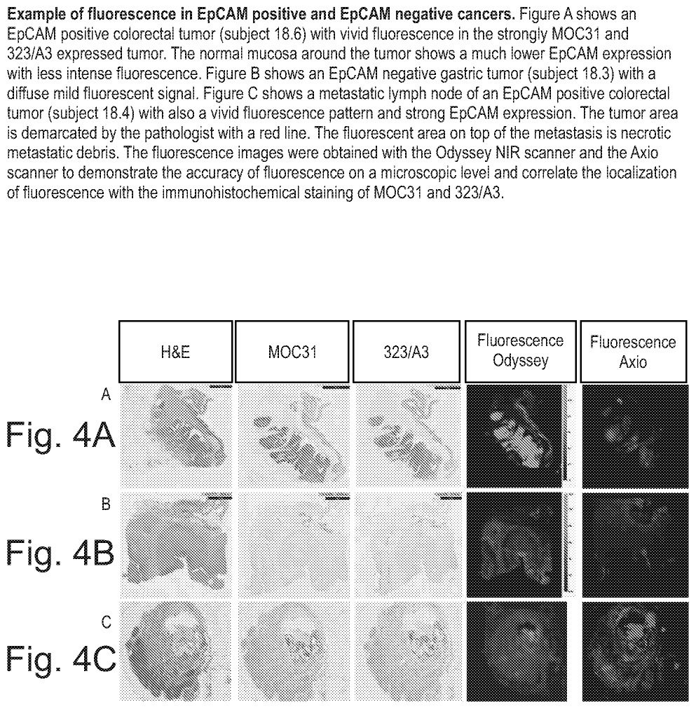

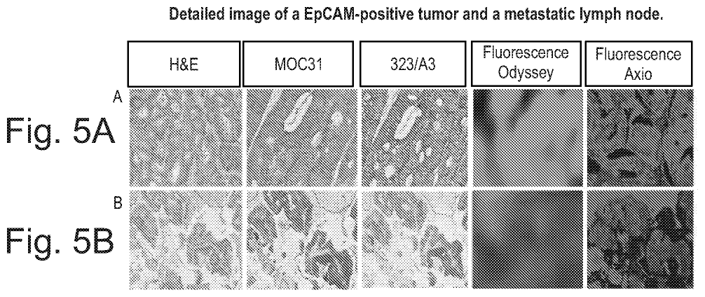

All resected tumor specimens were evaluated for EpCAM expression using immunohistochemistry with antibodies MOC31 and 323/A3. All the lower GI cancers showed strong MOC31 and 323/A3 staining (TIS 9-12) and emitted fluorescence with an ex vivo TBR of almost 2, with one TBR of 4.5 (median 1.9; range 1.7-4.5) (FIGS. 3A and 3B). The upper GI cancers showed variable EpCAM expressions (TIS range 0-12), where MOC31 and 323/A3 staining intensities ranged from negative to strong (FIGS. 3A and 3B). As would be predicted, both PCR cases (subject 18.2 with esophageal cancer and 18.7 with gastric cancer) showed a negative TIS and no fluorescence (TBR 1.0). One gastric tumor with vital malignant cells (subject 18.3) had a negative TIS, but an ex vivo TBR of 2.4, while the other gastric cancer (subject 6.4) had an ex vivo TBR of 1.9 with moderate 323/A3 and strong MOC31 staining. The esophageal tumors with vital malignant cells had mild to strong MOC31 and 323/A3 staining and ex vivo TBRs of almost 2.0 (range 1.8-1.9). FIG. 4A-C and FIG. 5A-5B shows an example of fluorescence images obtained with the Odyssey NIR scanner of an EpCAM-positive and EpCAM-negative tumor.

### Ex-Vivo Evaluation of Lymph Nodes

A total of 174 lymph nodes were evaluated for tumor, MOC31 and 323A3/expression and fluorescence. Out of 174 lymph nodes analyzed, 8 were tumor positive. A total of 6 metastatic lymph nodes were found in 2 patients in the low-dose cohort (n=3 for patient 6.3 and n=3 for patient 6.4), and 2 metastatic lymph nodes in the high-dose cohort (n=1 for patient 18.1 and n=1 for patient 18.5). The largest and most solid metastasis measured 3 mm over the longest axis. Seven out of 8 (87.5%) metastatic lymph nodes derived from poorly cohesive gastric or esophageal carcinomas and contained diffusely spread tumor cells or small cell clusters. The metastatic cell clusters demarcated by the pathologist on the HE-stained sections all showed EpCAM expression, by means of positive MOC31 and/or 323/A3 staining (FIG. 4A-C and FIG. 5A-5B). The TIS-scores of these metastatic areas were similar to those of the corresponding primary tumors. However, some individual tumor cells of approximately 10 μm marked by the pathologist on the HE-stained or IHC-stained sections were often not present in subsequent sections, presumably due to their small size. In some cases, this could hamper identification of individual cells in multiple subsequent sections for assessment of concordance between tumor status, IHC-staining and fluorescence. Nonetheless, this was not a problem with larger cell-clusters as these were visible in every microscopic section.

Assessment of the high-definition Axio scans showed a distinct fluorescent epithelial pattern in the metastatic lymph node area in a patient with well-differentiated colorectal carcinoma (FIG. 4A-C and FIG. 5A-5B). Combined with the detailed overlap of EpCAM expression observed in the IHC-stained sections, it clearly showed the targeting of VB5-845D-800CW to the EpCAM-positive tumor cells.

### Discussion

This study evaluated the safety and feasibility of VB5-845D-800CW for the detection and visualization of GI tumors during surgery. The study demonstrated a successful clinical translation, where VB5-845D-800CW up to 18 mg was well tolerated in 28 subjects (16 healthy volunteers and 12 patients) without signs of toxicity or hypersensitivity reactions. VB5-845D-800CW enabled adequate ex vivo tumor detection of both lower and upper GI cancers. Real-time intraoperative tumor detection was only possible during open oncologic resections and not during minimal invasive surgery, due to a lack of sensitivity of the NIR minimal invasive camera systems. Regarding open surgery, no clear differences in TBRs were apparent with varying dose levels when administered 3-4 days prior to surgery. However, when a longer injection time window of 5-6 days was implemented, a higher TBR was perceived. The preferential retention at the tumor site through specific binding combined with the loss of non-specific binding to normal tissue may explain the higher TBR with the longer time frame prior to assessment.

Microscopic evaluation of metastatic lymph nodes showed recognizable fluorescence patterns of tumor areas as small as 100 μm with EpCAM expression. The morphologic appearance of fluorescent epithelium in EpCAM-positive primary tumors and metastatic lymph nodes shows an adequate drug delivery to EpCAM-positive tumor cells.

Demonstrating safety of a novel fluorescent agent is of paramount importance in first-in-human trials as fluorescent imaging agents are commonly administered in vulnerable patient populations (i.e. oncologic patients) for the enhancement of tissue or vital structures. None of the included healthy volunteers nor patients in this study experienced AEs that were directly related to VB5-845D-800CW. Furthermore, the PK profile of VB5-845D-800CW established in healthy volunteers was valuable in designing the subsequent feasibility study in patients, as the dose range and approximate injection time window were determined for further appraisal. Preliminary PK analysis are tremendously resourceful as the apparent half-life and clearance rates can be estimated. The timing of administration prior to surgery and clearance patterns of an agent play important roles in optimizing fluorescence. It is often thought that an increase in dose results in improved fluorescence. This is partly true, as absolute tumor fluorescence increases in higher doses, but the background fluorescence (normal mucosa) increases consecutively, resulting in similar TBRs within different doses. This was apparent in the study, as well as in the preceding preclinical study in mice, where VB5-845D-800CW dose increases resulted in higher absolute tumor and background fluorescence, but not in higher TBRs (27). In the preclinical tumor models, the mean TBR increased significantly between 24 h and 72 h, due to a decrease in the absolute background signals (27). Antibodies (˜150 kDa) and Fab fragments (˜55 kDa) are known to have more complex PK and longer imaging lead times, when compared to smaller molecules (31-33). This was also confirmed in the PK analysis which showed an apparent half-life of 73 h in the dose 18 mg VB5-845D-800CW.

An unfortunate finding in this study was the discrepancy of intraoperative tumor fluorescence between open and minimal invasive procedures. No intraoperative fluorescence was seen in any of the minimal invasive procedures, while fluorescence was observable on the back table, caused by differences in camera sensitivity between the open camera head and minimal invasive camera. Experience with previous studies already uncovered that fluorescence during open surgery is superior to the minimal invasive setting, which can be explained by the decreased light illumination in laparoscopic devices, or the lower NIR light retrieval of the laparoscopic system. Since the decreased sensitivity is a known phenomenon in minimal invasive laparoscopes, back table imaging is incorporated in the surgical setting for additional fluorescence assessments. When the dose of 6 mg did not exhibit any fluorescence with the laparoscope, it was hypothesized that this dose was possibly too low for the minimal invasive setting. However, after dose escalation, the same distinctiveness was seen with 18 mg, while back table imaging showed adequate tumor fluorescence, confirming the lack of sensitivity of NIR laparoscopes.

Despite the lack of laparoscope sensitivity, the study empowered tumor fluorescence during open surgery of colorectal, gastric and oesophageal cancer with clear tumor demarcation ex vivo. Remarkably, it also disclosed no fluorescence in pCR cases, plausibly signifying that VB5-845D-800CW is tumor-specific, as it did not bind to chemoradiotherapy-induced fibrosis. This stipulates that VB5-845D-800CW may be a promising tool for the management of cancer patients as it can play a role in the endoscopic surveillance of rectal and oesophageal cancer patients eligible for the Watch-and-Wait (W&W) strategy after neoadjuvant therapy (34). The W&W strategy was implemented as an organ-preserving approach to decrease morbidity and improve long-term quality of life by preventing unnecessary surgery in patients with a clinical complete response after neoadjuvant therapy (34-38). In rectal cancer, local tumor regrowth rates occur in up to 25% of patients, where it is almost exclusively situated within the bowel wall (8). In oesophageal cancer, chemoradiotherapy with and without surgery are both widely accepted therapeutic approaches for the curative treatment of locally advanced oesophageal cancer. A prospective randomized controlled trial (39) showed that the addition of surgery after chemoradiotherapy, improves local tumor control but doesn't increase the survival rate as this is similar between patients receiving chemoradiotherapy and surgery or chemoradiotherapy alone. These findings make it sensible that NIR endoscopy with VB5-845D-800CW could be a safe and beneficial value in monitoring tumor regrowths (20, 35, 40, 41) in rectal and esophageal cancer patients. In addition, EpCAM could be useful in the endoscopic screening methods for gastric and esophageal cancers to help diagnose these tumors in an earlier stage. More than 70% of the gastric cancers are diagnosed in an advanced-stage due to a lack of specific clinical signs of early gastric cancer, also clarifying the dull prognosis of this cancer type (9). Hence, screening techniques play an important role in earlier detection for improved outcomes and mortality rates (9, 42). Alternative techniques have previously been suggested to improve the performance of endoscopy for better detection of pre-cancerous gastric or esophageal lesions, such as chromoendoscopy or the use of an anti-peristaltic agent (42, 43). NIR endoscopy with VB5-845D-800CW could also aid in this respect to accentuate (pre)cancerous lesions that are difficult to identify with conventional endoscopy (9).

For the evaluation of EpCAM, two antibodies against epitopes in the EGF-like domain I were used for immunohistochemistry; MOC31 and 323/A3 (44, 45). Both these antibodies react with different epitopes in the same extracellular domain on EpCAM, so similar immunohistochemistry reactivity would be expected. The highest EpCAM expression patterns were found in the lower GI cancers, which all showed strong staining of MOC31 and 323/A3. Moreover, immunohistochemistry confirmed EpCAM overexpression was preserved in the colorectal cancers after neoadjuvant therapy, conform preclinical results (26). The upper GI cancers showed more variety and different degrees of EpCAM expressions, which were not consistent. An interesting finding was the variance of EpCAM expression in the three included gastric cancers. All three gastric cancers were the diffuse type according to the Lauren classification, which have higher EpCAM expressions in comparison to the intestinal type. However, two of these gastric cancers had a negative TIS for MOC31 and 323/A3. Besides the Lauren classification, studies revealed EpCAM overexpression is also often related to the pathophysiology of gastric cancer, such as tumor size and lymph node metastasis (46). Patients with EpCAM overexpression exhibited a lower 5-year overall survival rate than EpCAM-negative patients (46). According to pathology reports, both the EpCAM-negative patients had no lymph node metastasis. Conversely, the third gastric patient had moderate to strong MOC31 and 323/A3 staining patterns and metastasized cancer in multiple lymph nodes. This finding suggests a possible relationship between EpCAM and the pathophysiology of gastric cancer, perhaps clarifying the low EpCAM expression in the two non-metastasized cancers. Nevertheless, one of the EpCAM-negative patients had a pCR with a corresponding ex vivo TBR of 1.0 (no fluorescence). The other EpCAM negative patient had an ex vivo TBR greater than 2. Fluorescence in this tumor could have been manifested by the enhanced permeability and retention (EPR) effect.

No conclusions could be made in this study regarding EpCAM expression and fluorescence, particularly in the upper GI cancers, and should be further investigated in a subsequent study.

Limitations of the study are the relatively low patient numbers (n=12) and the presence of numerous variable factors, such as dose, injection time window and tumor type. Due to the small patient numbers, an adequate comparison between these different factors could not be made. Despite of our findings regarding the adequate binding of VB45-845D-800CW to EpCAM-expressing tumor cells and cell-clusters within lymph nodes, we expect in vivo fluorescent imaging to be valuable for lymph nodes with larger (>5 mm), solid metastases. This is implied by the reported penetration depth of fluorescent signal which is approximately 5 millimeters, which could hamper detection of small cell-clusters surrounded by normal non-fluorescent lymph node tissue and adjacent fatty tissue or mesenteric structures (11).

Nevertheless, this phase I/II study was able to demonstrate safety and reveal that VB5-845D-800CW is a feasible and promising fluorescent agent for the detection of colorectal, gastric and esophageal cancer, but should be optimized in a larger subsequent follow-up study to validate the optimal dose and injection time window. The colorectal cancers consistently showed high EpCAM expressions, allegedly making it the most recommended tumor type for further evaluation. Nonetheless, to our knowledge this is the first study to investigate a tumor-targeted fluorescent agent in gastric and esophageal cancers, which showed encouraging results for further assessment in upper GI cancers. It would be interesting to evaluate a possible relationship between EpCAM expression and the pathophysiology of gastric cancers (i.e. presence of metastatic lymph nodes and Lauren classification) and effect on fluorescence. This pilot study forms an encouraging platform for further imaging studies with VB5-845D-800CW, as it can help surgeons identify GI tumors in real-time, potentially be valuable in reducing residual tumor burden and be a helpful tool in screening programs or W&W strategies after neoadjuvant therapy.

### Example 3: Liquid Biopsy

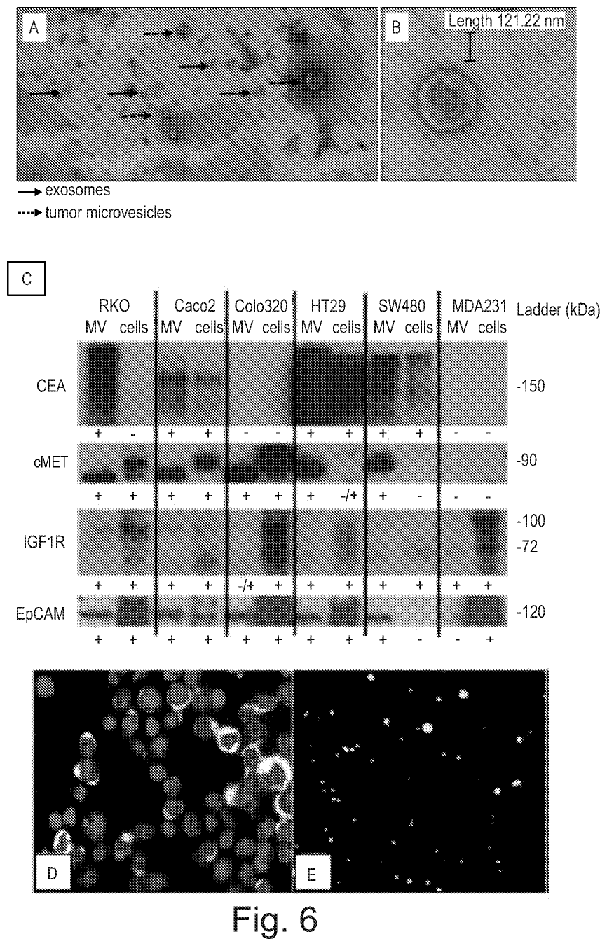

Blood from a healthy male donor was spiked with human colonic cancer cells and pre-isolated extracellular vesicles (EV). The EVs were derived from various human colon cancer cell lines and cultured in medium with EV-depleted fetal calf serum. The results are illustrated in FIG. 6A-E, showing the presence of cancer cells and vesicles derived from blood by simple fluorescence microscopy.

### REFERENCES

1. Parkin D M, Bray F, Ferlay J, Pisani P. Global cancer statistics, 2002. CA Cancer J Clin. 2005; 55(2):74-108.
2. Wu H, Wang W, Tong S, Wu C. Nucleostemin regulates proliferation and migration of gastric cancer and correlates with its malignancy. Int J Clin Exp Med. 2015; 8(10):17634-43.
3. Hohenberger W, Weber K, Matzel K, Papadopoulos T, Merkel S. Standardized surgery for colonic cancer: complete mesocolic excision and central ligation—technical notes and outcome. Colorectal Dis. 2009; 11(4):354-64; discussion 64-5.
4. Maas M, Nelemans P J, Valentini V, Das P, Rodel C, Kuo L J, et al. Long-term outcome in patients with a pathological complete response after chemoradiation for rectal cancer: a pooled analysis of individual patient data. Lancet Oncol. 2010; 11(9):835-44.
5. Al-Sukhni E, Attwood K, Mattson D M, Gabriel E, Nurkin S J. Predictors of Pathologic Complete Response Following Neoadjuvant Chemoradiotherapy for Rectal Cancer. Ann Surg Oncol. 2016; 23(4):1177-86.
6. Coccolini F, Nardi M, Montori G, Ceresoli M, Celotti A, Cascinu S, et al. Neoadjuvant chemotherapy in advanced gastric and esophago-gastric cancer. Meta-analysis of randomized trials. Int J Surg. 2018; 51:120-7.
7. Miao Z F, Liu X Y, Wang Z N, Zhao T T, Xu Y Y, Song Y X, et al. Effect of neoadjuvant chemotherapy in patients with gastric cancer: a PRISMA-compliant systematic review and meta-analysis. BMC Cancer. 2018; 18(1):118.
8. van der Valk M J M, Hilling D E, Bastiaannet E, Meershoek-Klein Kranenbarg E, Beets G L, Figueiredo N L, et al. Long-term outcomes of clinical complete responders after neoadjuvant treatment for rectal cancer in the International Watch & Wait Database (IWWD): an international multicentre registry study. Lancet. 2018; 391(10139):2537-45.
9. Song Z, Wu Y, Yang J, Yang D, Fang X. Progress in the treatment of advanced gastric cancer. Tumour Biol. 2017; 39(7):1010428317714626.
10. Olson M T, Ly Q P, Mohs A M. Fluorescence Guidance in Surgical Oncology: Challenges, Opportunities, and Translation. Mol Imaging Biol. 2019; 21(2):200-18.
11. Vahrmeijer A L, Hutteman M, van der Vorst J R, van de Velde C J, Frangioni J V. Image-guided cancer surgery using near-infrared fluorescence. Nat Rev Clin Oncol. 2013; 10(9):507-18.
12. Boogerd L S, Handgraaf H J, Lam H D, Huurman V A, Farina-Sarasqueta A, Frangioni J V, et al. Laparoscopic detection and resection of occult liver tumors of multiple cancer types using real-time near-infrared fluorescence guidance. Surg Endosc. 2017; 31(2):952-61.
13. Schaafsma B E, Mieog J S, Hutteman M, van der Vorst J R, Kuppen P J, Lowik C W, et al. The clinical use of indocyanine green as a near-infrared fluorescent contrast agent for image-guided oncologic surgery. J Surg Oncol. 2011; 104(3):323-32.
14. Boogerd L S F, Hoogstins C E S, Schaap D P, Kusters M, Handgraaf H J M, van der Valk M J M, et al. Safety and effectiveness of SGM-101, a fluorescent antibody targeting carcinoembryonic antigen, for intraoperative detection of colorectal cancer: a dose-escalation pilot study. Lancet Gastroenterol Hepatol. 2018; 3(3):181-91.
15. Hoogstins C E S, Boogerd L S F, Sibinga Mulder B G, Mieog J S D, Swijnenburg R J, van de Velde C J H, et al. Image-Guided Surgery in Patients with Pancreatic Cancer: First Results of a Clinical Trial Using SGM-101, a Novel Carcinoembryonic Antigen-Targeting, Near-Infrared Fluorescent Agent. Ann Surg Oncol. 2018; 25(11):3350-7.
16. Hoogstins C E, Tummers Q R, Gaarenstroom K N, de Kroon C D, Trimbos J B, Bosse T, et al. A Novel Tumor-Specific Agent for Intraoperative Near-Infrared Fluorescence Imaging: A Translational Study in Healthy Volunteers and Patients with Ovarian Cancer. Clin Cancer Res. 2016; 22(12):2929-38.
17. Ishizawa T, Bandai Y, Ijichi M, Kaneko J, Hasegawa K, Kokudo N. Fluorescent cholangiography illuminating the biliary tree during laparoscopic cholecystectomy. Br J Surg. 2010; 97(9):1369-77.
18. de Valk K S, Handgraaf H J, Deken M M, Sibinga Mulder B G, Valentijn A R, Terwisscha van Scheltinga A G, et al. A zwitterionic near-infrared fluorophore for real-time ureter identification during laparoscopic abdominopelvic surgery. Nat Commun. 2019; 10(1):3118.
19. Schaap D P, de Valk K S, Deken M M, Meijer R P J, Burggraaf J, Vahrmeijer A L, et al. Carcinoembryonic antigen-specific, fluorescent image-guided cytoreductive surgery with hyperthermic intraperitoneal chemotherapy for metastatic colorectal cancer. Br J Surg. 2020.
20. Tjalma J J J, Koller M, Linssen M D, Hartmans E, de Jongh S, Jorritsma-Smit A, et al. Quantitative fluorescence endoscopy: an innovative endoscopy approach to evaluate neoadjuvant treatment response in locally advanced rectal cancer. Gut. 2020; 69(3):406-10.
21. Patriarca C, Macchi R M, Marschner A K, Mellstedt H. Epithelial cell adhesion molecule expression (CD326) in cancer: a short review. Cancer Treat Rev. 2012; 38(1):68-75.
22. Spizzo G, Fong D, Wurm M, Ensinger C, Obrist P, Hofer C, et al. EpCAM expression in primary tumour tissues and metastases: an immunohistochemical analysis. J Clin Pathol. 2011; 64(5):415-20.
23. Went P T, Lugli A, Meier S, Bundi M, Mirlacher M, Sauter G, et al. Frequent EpCam protein expression in human carcinomas. Hum Pathol. 2004; 35(1):122-8.
24. Spizzo G, Obrist P, Ensinger C, Theurl I, Dunser M, Ramoni A, et al. Prognostic significance of Ep-CAM AND Her-2/neu overexpression in invasive breast cancer. Int J Cancer. 2002; 98(6):883-8.
25. Went P, Vasei M, Bubendorf L, Terracciano L, Tornillo L, Riede U, et al. Frequent high-level expression of the immunotherapeutic target Ep-CAM in colon, stomach, prostate and lung cancers. Br J Cancer. 2006; 94(1):128-35.
26. Boogerd L S, van der Valk M J, Boonstra M C, Prevoo H A, Hilling D E, van de Velde C J, et al. Biomarker expression in rectal cancer tissue before and after neoadjuvant therapy. Onco Targets Ther. 2018; 11:1655-64.
27. Boogerd L S F, Boonstra M C, Prevoo H, Handgraaf H J M, Kuppen P J K, van de Velde C J H, et al. Fluorescence-guided tumor detection with a novel anti-EpCAM targeted antibody fragment: Preclinical validation. Surg Oncol. 2019; 28:1-8.
28. van Driel P B, Boonstra M C, Prevoo H A, van de Giessen M, Snoeks T J, Tummers Q R, et al. EpCAM as multi-tumour target for near-infrared fluorescence guided surgery. BMC Cancer. 2016; 16(1):884.
29. Wang Y Y, Li L, Zhao Z S, Wang Y X, Ye Z Y, Tao H Q. L1 and epithelial cell adhesion molecules associated with gastric cancer progression and prognosis in examination of specimens from 601 patients. J Exp Clin Cancer Res. 2013; 32:66.
30. Hoogstins C, Burggraaf J J, Koller M, Handgraaf H, Boogerd L, van Dam G, et al. Setting Standards for Reporting and Quantification in Fluorescence-Guided Surgery. Mol Imaging Biol. 2019; 21(1):11-8.
31. Hernot S, van Manen L, Debie P, Mieog J S D, Vahrmeijer A L. Latest developments in molecular tracers for fluorescence image-guided cancer surgery. Lancet Oncol. 2019; 20(7):e354-e67.
32. Wittrup K D, Thurber G M, Schmidt M M, Rhoden J J. Practical theoretic guidance for the design of tumor-targeting agents. Methods Enzymol. 2012; 503:255-68.
33. Freise A C, Wu A M. In vivo imaging with antibodies and engineered fragments. Mol Immunol. 2015; 67(2 Pt A):142-52.
34. Beets G L, Figueiredo N F, Beets-Tan R G. Management of Rectal Cancer Without Radical Resection. Annu Rev Med. 2017; 68:169-82.
35. Renehan A G, Malcomson L, Emsley R, Gollins S, Maw A, Myint A S, et al. Watch-and-wait approach versus surgical resection after chemoradiotherapy for patients with rectal cancer (the OnCoRe project): a propensity-score matched cohort analysis. Lancet Oncol. 2016; 17(2):174-83.
36. Breugom A J, van de Velde C J. Is it time for watchful waiting for rectal cancer? Lancet Oncol. 2015; 16(8):875-6.
37. van der Valk M J M, Hilling D E, van de Velde C J H. What is there to learn for watch-and-wait in rectal cancer? Lancet Gastroenterol Hepatol. 2017; 2(7):467-8.
38. Wang J, Qin J, Jing S, Liu Q, Cheng Y, Wang Y, et al. Clinical complete response after chemoradiotherapy for carcinoma of thoracic esophagus: Is esophagectomy always necessary? A systematic review and meta-analysis. Thorac Cancer. 2018; 9(12):1638-47.
39. Stahl M, Stuschke M, Lehmann N, Meyer H J, Walz M K, Seeber S, et al. Chemoradiation with and without surgery in patients with locally advanced squamous cell carcinoma of the esophagus. J Clin Oncol. 2005; 23(10):2310-7.
40. Dossa F, Chesney T R, Acuna S A, Baxter N N. A watch-and-wait approach for locally advanced rectal cancer after a clinical complete response following neoadjuvant chemoradiation: a systematic review and meta-analysis. Lancet Gastroenterol Hepatol. 2017; 2(7):501-13.
41. Martens M H, Maas M, Heijnen L A, Lambregts D M, Leijtens J W, Stassen L P, et al. Long-term Outcome of an Organ Preservation Program After Neoadjuvant Treatment for Rectal Cancer. J Natl Cancer Inst. 2016; 108(12).
42. Domper Arnal M J, Ferrandez Arenas A, Lanas Arbeloa A. Esophageal cancer: Risk factors, screening and endoscopic treatment in Western and Eastern countries. World J Gastroenterol. 2015; 21(26):7933-43.
43. Yao K. The endoscopic diagnosis of early gastric cancer. Ann Gastroenterol. 2013; 26(1):11-22.
44. Balzar M, Briaire-de Bruijn I H, Rees-Bakker H A, Prins F A, Helfrich W, de Leij L, et al. Epidermal growth factor-like repeats mediate lateral and reciprocal interactions of Ep-CAM molecules in homophilic adhesions. Mol Cell Biol. 2001; 21(7):2570-80.
45. Winter M J, Nagtegaal I D, van Krieken J H, Litvinov S V. The epithelial cell adhesion molecule (Ep-CAM) as a morphoregulatory molecule is a tool in surgical pathology. Am J Pathol. 2003; 163(6):2139-48.
46. Dai M, Yuan F, Fu C, Shen G, Hu S, Shen G. Relationship between epithelial cell adhesion molecule (EpCAM) overexpression and gastric cancer patients: A systematic review and meta-analysis. PLoS One. 2017; 12(4):e0175357.

1. A molecule comprising an antibody or antibody fragment capable of specifically targeting EpCAM conjugated to a near-infrared (NIR) fluorescent dye.

2. The molecule of claim 1 wherein the antibody fragment is VB5-845D.

3. The molecule of claim 1, wherein the antibody or antibody fragment comprises a heavy chain having 90% sequence identity to amino acid SEQ ID NO: 1, 3 or 5 and a light chain having 90% sequence identity to amino acid SEQ ID NO: 2, 4 or 6.

4. The molecule of claim 1, wherein the NIR fluorescent dye is IRDye 800CW.

5. (canceled)

6. A composition comprising the molecule of claim 1 and a pharmaceutically acceptable excipient, carrier, buffer or stabilizer.

7. A method for intraoperative tumor visualization of EpCAM expressing primary tumors and/or lymph nodes, the method comprising intravenous administration to a patient prior to surgery an infusion solution comprising the molecule of claim 1.

8. The method according to claim 7, wherein the tumor is subsequently examined for the presence of EpCAM.

9. (canceled)

10. (canceled)

11. A method for tumor visualization of EpCAM expressing tumors, the method comprising the topical administration of the molecule of claim 1 or a composition comprising the molecule to a resected tumor.

12. The method according to claim 11, wherein the tumor is subsequently examined for the presence of EpCAM.

13. (canceled)

14. A method for tumor visualization of EpCAM expressing tumors, the method comprising administering the molecule of claim 1 or a composition comprising the molecule to the gastrointestinal tract.

15. The method according to claim 14, wherein the tumor is subsequently examined for the presence of EpCAM.

16. (canceled)

17. A method for visualization of EpCAM in circulating cells or fragments in blood, the method comprising the intravenous administration of the molecule of claim 1 or a composition comprising the molecule to a patient.

18. The method according to claim 17, wherein the blood is subsequently examined for the presence of EpCAM.

19. (canceled)

20. A method for removing EpCAM expressing tumor or tissue, comprising:
i) administering an infusion solution comprising the molecule of claim 1,
ii) waiting for a period of time to allow the solution to migrate into the tumor and/or tissue,
iii) visualizing the tumor or tissue for the presence of the molecule and
iv) removing the visualized tumor or tissue.

21. The method according to claim 20, wherein the tumor or tissue is visualised using fluorescence imaging.

22. A method for determining whether rectal and esophageal cancer patients after neoadjuvant therapy require surgery, comprising
i) administering to a patient an infusion solution comprising the molecule of claim 1,
ii) waiting for a period of time to allow the solution to migrate into the tissue, and
iii) performing endoscopic surveillance to identify whether there are any EpCAM expressing tumor cells present.

23. A method for detecting EpCAM expressing primary tumors and lymph nodes, by contacting a primary tumor or lymph node with the molecule of claim 1.

24. (canceled)

25. A method for diagnosing a disease or condition in a human or animal, by contacting a tissue of the human or animal with the molecule of claim 1.

26. (canceled)

27. (canceled)

28. (canceled)

29. (canceled)

30. A method for detecting cancer in a human or animal, by contacting a tissue from the human or animal with the molecule of claim 1, optionally wherein the cancer is lower gastro intestinal and/or upper gastro intestinal cancer.

31. (canceled)

32. (canceled)

33. (canceled)

34. (canceled)

35. A method for detecting EpCAM expressing circulating cells or fragments in blood, by contacting the blood with the molecule of claim 1.

36. (canceled)
